(19) United States
(12) Patent Application Publication
(10) Pub. No.: US 2013/0344512 A1
(43) Pub. Date: Dec. 26, 2013

(54) CD16A REPORTER ASSAY FOR EVALUATION OF ADCC POTENTIAL OF BIOLOGICS

(71) Applicants: Jose Miguel Aste-Amezaga, Harleysville, PA (US); Pamela K. Mathis, Collegeville, PA (US)

(72) Inventors: Jose Miguel Aste-Amezaga, Harleysville, PA (US); Pamela K. Mathis, Collegeville, PA (US)

(73) Assignee: MERCK SHARP & DOHME CORP, RAHWAY, NJ (US)

(21) Appl. No.: 14/002,898

(22) PCT Filed: Feb. 27, 2012
(86) PCT No.: PCT/US12/26681

§ 371 Date: Sep. 3, 2013

Related U.S. Application Data
(60) Provisional application No. 61/449,320, filed on Mar. 4, 2011.

(51) Int. Cl. G01N 33/50

(52) U.S. Cl. CPC G01N 33/5014
USPC 435/7.24; 435/325; 435/455; 435/7.21

(57) ABSTRACT

The present invention provides a method for determining whether an antibody or antigen-binding fragment thereof will cause ADCC when administered to a subject. Host cells that may be used in such a method are also provided.

This application claims the benefit of U.S. provisional patent application No. 61/449,320, filed Mar. 4, 2011; which is herein incorporated by reference in its entirety.

### FIELD OF THE INVENTION

The field of the invention relates to methods for evaluating ADCC associated with antibodies and antigen-binding fragments thereof.

### BACKGROUND OF THE INVENTION

Laboratory methods exist for determining the efficacy of antibodies or effector cells in eliciting ADCC. Among these methods include chromium-51 [⁵¹Cr] release assay, europium [Eu] release assay, and sulfur-35 [³⁵S] release assay. Usually, a labeled target cell line expressing a certain surface-exposed antigen is incubated with antibody specific for that antigen. After washing, effector cells expressing Fc receptor CD16 are co-incubated with the antibody-bound, labeled target cells. Target cell lysis is subsequently measured by release of intracellular label by a scintillation counter or spectrophotometry. These assays are cumbersome and may involve the use of radioisotopes.

### SUMMARY OF THE INVENTION

The assays of the present invention measure the potential antibody-dependent cellular cytotoxicity (ADCC) of therapeutic monoclonal antibodies and Fc-fusion proteins. Unlike traditional ADCC protocols which measure cell lysis, the assays of the present invention quantify signal transduction by CD16A-FcεR1γ following engagement by an antibody bound either to its target antigen on cells or to recombinantly expressed target antigen e.g., immobilized on a solid substrate.

The present invention provides an isolated host cell (e.g., a T-lymphocyte, an immortalized T lymphocyte, a Jurkat cell or a Wil-2 B-cell) comprising CD16A, or a functional variant thereof, fused to FcεR1γ, or a functional variant thereof (e.g., CD16A^{158V}-FcεR1γ), wherein the fusion is bound to the Fc domain of an antibody or antigen-binding fragment thereof that is complexed with an antigen that is either expressed on the surface of a cell or immobilized to a substrate, such as, for example, VEGFR, IGF1R, RANK, RANKL, or tumor necrosis factor alpha precursor, and a polynucleotide comprising a promoter that comprises one or more NFAT responsive elements, operably linked to a reporter gene (e.g., beta-lactamase gene). A method for making such a host cell is also provided, which method comprises introducing a polynucleotide encoding the fusion and the polynucleotide comprising the promoter operably linked to the reporter gene into an isolated host cell and culturing the host cell under conditions wherein the fusion is expressed and located on the surface of the cell.

The present invention also provides a method for evaluating the potential for antibody-dependent cellular cytotoxicity of an antibody or antigen-binding fragment thereof when administered to a subject comprising contacting a cell (a T-lymphocyte, an immortalized T lymphocyte, a Jurkat cell or a Wil-2 B-cell) expressing CD16A fused to FcεR1γ (e.g., CD16A^{158V}-FcεR1γ) with a complex between an antibody or antigen-binding fragment thereof and an isolated antigen (e.g., VEGFR, IGF1R, RANK, RANKL, or tumor necrosis factor alpha precursor); and measuring CD16A-mediated expression of a reporter gene (e.g., beta-lactamase gene) operably linked to a promoter comprising one or more NFAT responsive elements in said cell; wherein the antibody or fragment is determined to exhibit said cytotoxicity if said expression is observed. For example, wherein the method includes (1) introducing, into an isolated host cell: (i) a polynucleotide comprising a promoter that comprises one or more NFAT responsive elements, operably linked to a reporter gene; and (ii) a polynucleotide encoding a CD16A-FcεR1γ fusion protein which is operably linked to a promoter; wherein the fusion, when on the host cell surface, is capable of interacting with an antibody or antigen-binding fragment thereof complexed with an antigen; (2) exposing the host cell to an antibody or antigen-binding fragment thereof complexed with an antigen; and (3) determining if expression of the reporter gene is activated; wherein the antibody or antigen-binding fragment thereof is determined to cause said cytotoxicity if said expression is observed. For example, wherein the reporter gene is beta-lactamase and wherein beta-lactamase reporter gene activation is detected by adding CCF2-AM substrate to said isolated host cell comprising the reporter gene and determining whether said host cell fluoresces light having a wavelength in the range of about 460 nm to about 530 nm when excited with light of a wavelength of about 409 nm; wherein the antibody or fragment is determined to cause said cytotoxicity if said fluorescence is detected.

### BRIEF DESCRIPTION OF THE FIGURES

FIG. 1 (A & B). Differential activation by trastuzumab and variants in CD16A Reporter Assays (cell-based and cell-free formats); (A) Target (Her2) SKOV3 cell-based format; (B) Target (HER2-ECD) cell-free antigen format.

FIG. 2 (A & B). Differential activation by infliximab, infliximab variant and etanercept in CD16A Reporter Assay (cell-based and cell-free formats); (A) Target (TNFalpha HEK 293Flpln/TNF(alpha) delta1-12 cell-based format; (B) Target (huTNFalpha protein) cell-free antigen format.

FIG. 3. Differential activation by rituxumab and variants in CD16A Reporter Assay (cell-based format):Target (CD20) Raji cell-based format.

FIG. 4 (A & B). Comparison of classical ADCC and CD16A Reporter Assay using infliximab and etanercept as a model; (A) Classical ADCC assay using JurkatFlpln/TNF(alpha) delta1-12 as target cells and primary natural killer (NK) cells isolated from whole blood as effector cells; (B) CD16 Reporter assay using HEK 293Flpln/TNF(alpha) delta1-12 as target cells and Jurkat/NFat-bla/CD16A as effector cells.

### DETAILED DESCRIPTION OF THE INVENTION

The assays of the present invention include determining whether a reporter gene is expressed in a host cell to which an antibody/antigen complex binds via a CD16A-FcεR1γ fusion expressed on the surface of the host cell. The reporter gene is operably linked to a promoter comprising one or more NFAT responsive elements which mediate NFAT-dependent transcription of the reporter gene. Binding of the antibody/antigen complex, wherein the antigen is located on a cell surface or is not cell bound but is immobilized on a solid substrate, to the fusion signals to the promoter/reporter construct and, thereby, causes the reporter gene transcription.

“Antibody-Dependent Cell-Mediated Cytotoxicity” or “ADCC” is a mechanism of cell-mediated immunity whereby an effector cell (e.g., a natural killer (NK) cell; neutrophil or eosinophil) actively lyses a target cell that has been bound by antibodies.

An “antigen-binding fragment” of an antibody includes both an antigen-binding site and an FC domain that is capable of binding an Fc receptor.

An antigen is located or expressed “on” a cell surface (e.g., cell membrane) if it is physically associated with the outer surface of a cell or is embedded in the outer cell surface (e.g., partially) or is physically associated with any protein that, itself, is physically associated with the outer surface of a cell or is embedded in the outer cell surface (e.g., partially). Examples of antigens on a cell surface include cell surface receptors.

In an embodiment of the invention, an NFAT responsive element is 5′-ggaggaaaaa ctgtttcatacagaaaggcgt-3′ (SEQ ID NO: 1) or any variant thereof having, e.g., 1, 2, 3, 4 or 5 substitutions, which can still promote NFAT-dependent transcription. The NFAT responsive element may appear, for example, in the context of any promoter that can promoter transcription of a gene to which it is operably linked upon binding of the NFAT transcription factor to the NFAT responsive element. In an embodiment of the invention, the promoter includes one or more NFAT responsive elements, e.g., a tandem repeat of NFAT responsive elements. Examples of promoters in which an NEAT responsive element may be located and which may be operably linked to a reporter gene include, but are not limited to, a CMV promoter, e.g., a minimal CMV promoter. In an embodiment of the invention, the promoter comprising the NFAT responsive element(s) and a reporter gene to which it is operably linked is stably integrated into the chromosomal DNA of an isolated host cell or is episomal in the host cell, e.g., in a plasmid.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A polypeptide or protein comprises two or more amino acids.

The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, (4) was isolated or purified e.g., by a technician and/or (5) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A “polynucleotide”, “nucleic acid” or “nucleic acid molecule” includes double-stranded and single-stranded DNA and RNA.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

An amino acid sequence comprises two or more amino acids.

A “coding sequence” or a sequence “encoding” an expression product, such as an RNA or polypeptide, is a nucleotide sequence that, when expressed, results in production of the product.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A “promoter” or “promoter sequence” includes a promoter that can cause NFAT-dependent transcription of a gene to which it is operably linked (e.g., a reporter gene) in an isolated host cell that comprises the promoter and gene, e.g., wherein the promoter comprises one or more NFAT responsive elements. Promoters include the cytomegalovirus (CMV) promoter (U.S. Pat. Nos. 5,385,839 and 5,168,062), e.g., a minimal CMV promoter, the SV40 early promoter region (Benoist, et al., (1981) Nature 290:304-310), the promoter contained in the 3′ long terminal repeat of Rous sarcoma virus (Yamamoto, et al, (1980) Cell 22:787-797), the herpes thymidine kinase promoter (Wagner, et al., (1981) Proc. Natl. Acad. Sci. USA 78:1441-1445).

A coding sequence, such as a reporter gene, is “under the control of”, “functionally associated with” or “operably linked to” a transcriptional and translational control sequence, such as a promoter, e.g., in an isolated host cell, when the sequences direct RNA polymerase mediated transcription of the coding sequence into RNA, e.g., mRNA, which then may be trans-RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The terms “vector”, “cloning vector” and “expression vector” mean the vehicle (e.g., a plasmid) by which a DNA or RNA sequence can be introduced into a host cell, so as to transform the host and, optionally, promote expression and/or replication of the introduced sequence.

The term “transformation” means the introduction of a nucleic acid into a cell. These terms may refer to the introduction of a nucleic acid encoding CD16A-FcεR1γ into a cell. The introduced gene or sequence may be called a “clone”. A host cell that receives the introduced DNA or RNA has been “transformed” and is a “transformant” or a “clone”. The DNA or RNA introduced to a host cell can come from any source, including cells of the same genus or species as the host cell, or cells of a different genus or species.

Host cells that can be used in a screening assay of the present invention include any cell that can express a CD16A-FcεR1γ fusion and, when exposed to an antigen/antibody or antigen-binding fragment thereof complex wherein the antibody or fragment that has the potential to cause ADCC upon antigen binding, causes NFAT-dependent expression of a reporter gene that is operably linked to a promoter comprising one or more NFAT responsive elements. Specific examples of such cells include T-lymphocytes, e.g., immortalized T lymphocytes such as Jurkat cells (e.g., deposited at the American Type Culture Collection (ATCC) under number TIB-152); NFAT-bla Jurkat cell; Wil-2 B-cells (ATCC #CRL-8885); or Raji cells.

### CD16A-FcεR1γ

The present invention provides an isolated fusion polypeptide comprising human CD16A or a functional variant thereof fused to FcεR1γ or a functional variant thereof, isolated host cells (e.g., host cells that are discussed herein) comprising the fusions (e.g., bound to an antibody or antigen-binding fragment thereof/antigen complex) and methods of use thereof, e.g., as is discussed herein.

The human CD16A gene and polypeptide are very well known in the art. In an embodiment of the invention, human CD16A comprises the amino acid sequence:

(SEQ ID NO: 2)
MWQLLLPTALLLLVSAGMRTEDLPKAVVFLEPQWYRVLEKDSVTLKCQGAYSPEDNSTQWFHNESLISSQASSYFID
AATVDDSGEYRCQTNLSTLSDPVQLEVHIGWLLLQAPRWVFKEEDPIHLRCHSWKNTALHKVTYLQNGKGRKYFHHN
SDFYIPKATLKDSGSYFCRGLVGSKNVSSETVNITITQGLAVSTISSFFPPGEF

In an embodiment of the invention, the CD16A comprises the 158V polymorphism (as set forth above); in another embodiment of the invention, the CD16A comprises the 158F polymorphism wherein the bold, underscored residue in the sequence set forth above, in SEQ ID NO: 2, is F.

The human Fc epsilon RI gamma or FcεR1γ gene and polypeptide are also well known in the art. In an embodiment of the invention, FcεR1γ polypeptide comprises the amino acid sequence:

(SEQ ID NO: 3)
PQLCYILDAILFLYGIVLTLLYCRLKVIQVRKAAITSYEKSDGVYTGLSTRNQETYETLKHEKPPQ

In an embodiment of the invention, the CD16A-FcεR1γ fusion comprises the following amino acid sequence:

(SEQ ID NO: 4)
MWQLLLPTALLLLVSAGMRTEDLPKAVVFLEPQWYRVLEKDSVTLKCQGAYSPEDNSTQWFHNESLISSQASSYFID
AATVDDSGEYRCQTNLSTLSDPVQLEVHIGWLLLQAPRWVFKEEDPIHLRCHSWKNTALHKVTYLQNGKGRKYFHHN
SDFYIPKATLKDSGSYFCRGLVGSKNVSSETVNITITQGLAVSTISSFFPPGEFPQLCYILDAILFLYGIVLTLLYC
RLKVIQVRKAAITSYEKSDGVYTGLSTRNQETYETLKHEKPPQ

e.g., in an embodiment of the invention, the fusion is encoded by the nucleotide sequence:

(SEQ ID NO: 5)
agctctctggctaactagagaacccactgcttactggcttatcgaaattaatacgactcactata
gggagacccaagctggctagcgtttaaacttaagcttggtaccgagctcGGATCCCTTTGGTGAC
TTGTCCACTCCAGTGTGGCATCATGTGGCAGCTGCTCCTCCCAACTGCTCTGCTACTTCTAGTTT
CAGCTGGCATGCGGACTGAAGATCTCCCAAAGGCTGTGGTGTTCCTGGAGCCTCAATGGTACAGG
GTGCTCGAGAAGGACAGTGTGACTCTGAAGTGCCAGGGAGCCTACTCCCCTGAGGACAATTCCAC
ACAGTGGTTTCACAATGAGAGCCTCATCTCAAGCCAGGCCTCGAGCTACTTCATTGACGCTGCCA
CAGTCGACGACAGTGGAGAGTACAGGTGCCAGACAAACCTCTCCACCCTCAGTGACCCGGTGCAG
CTAGAAGTCCATATCGGCTGGCTGTTGCTCCAGGCOCCTCGGTGGGTGTTCAAGGAGGAAGACCC
TATTCACCTGAGGTGTCACAGCTGGAAGAACACTGCTCTGCATAAGGTCACATATTTACAGAATG
GCAAAGGCAGGAAGTATTTTCATCATAATTCTGACTTCTACATTCCAAAAGCCACACTCAAAGAC
AGCGGCTCCTACTTCTGCAGGGGGCTTGTTGGGAGTAAAAATGTGTOTTCAGAGACTGTGAACA
TCACCATCACTCAAGGTTTGGCAGTGTCAACCATCTCATCATTCTTTCCACCTGGGGAATTC
CCTCAGCTCTGCTATATCCTGGATGCCATCCTGTTTCTGTATGGAATTGTCCTCACCCTCCTC
TACTGTCGACTGAAGGTAATCCAAGTGCGAAAGGCAGCTATAACCAGCTATGAGAAATCAGA
TGGTGTTTACACGGGCCTGAGCACCAGGAACCAGGAGACTTACGAGACTCTGAAGCATGAG
AAACCACCACAGTAGGCGGCCGCtcgagtctaga

In an embodiment of the invention the fusion comprises the CD16A leader sequence, 206 residues of the CD16A extracellular domain, 2 residues of the FcεR1γ extracellular domain and 64 residues of the FcεR1γ transmembrane and intracellular domains.

### Screening Assays

The present invention provides, in part, methods for evaluating the potential for a given antibody or antigen-binding fragment thereof to mediate ADCC in the body of a subject (e.g., a mammal such as a mouse, rat, rabbit, primate or human) that is administered the antibody or fragment. Such methods evaluate the activation of the CD16A pathway in a cell in response to immunoglobulin binding. For example, the present invention provides a method for evaluating the potential for antibody-dependent cellular cytotoxicity of an antibody or antigen-binding fragment thereof when administered to a subject comprising contacting a cell line expressing CD16A (e.g., CD16A^{158V} or a functional variant thereof) fused with FcεR1γ or a functional variant thereof with a complex between an antibody or antigen-binding fragment thereof and an antigen; and measuring CD16A mediated transcriptional activation of NFAT from a promoter comprising 1 or more NFAT responsive elements in said cell. In an embodiment of the invention, the cell expresses CD16A-FcεR1γ, e.g., comprising the amino acid sequence set forth in SEQ ID NO: 4. In an embodiment of the invention, the methods of the present invention comprise binding the antigen which is on the surface of a cell or a cell membrane in a cellular fraction; or is immobilized on a solid substrate (e.g., glass, plastic, sepharose or agarose).

In an embodiment of the invention, antigen may be coated to cell culture dishes, multi-well plates for high-throughput assays; polymer beads, gold beads; lipid and other nanoparticles; soluble polymers; cell-derived vesicles (e.g., exosomes; RBC ghosts). Antigen coupling may be achieved via non-specific interactions (e.g., hydrophobic), non-covalent specific interactions (e.g., biotin-streptavidin), or covalent chemical conjugation. Antigen may be coated across the surface of individual wells or printed onto high-density arrays e.g., for single cell monitoring by imaging techniques.

CD16A-mediated transcriptional activation of a promoter comprising 1 or more NFAT responsive elements can be evaluated by any method known in the art. For example, the cell expressing the CD16A may comprise a promoter, including one or more NFAT responsive elements, that is operably linked to a reporter gene (e.g., beta-lactamase or a sequence not naturally operably linked to an NEAT responsive element). In such a case, CD16A-mediated transcriptional activation of the promoter comprising the NFAT responsive element(s) is correlated with reporter gene signal or reporter gene expression levels.

The present invention also provides a method for determining if a test antibody or antigen-binding fragment thereof causes antibody-dependent cell-mediated cytotoxicity (ADCC) upon binding of an antigen comprising:

(i) introducing, into an isolated host cell:
a polynucleotide comprising a promoter that comprises one or more NEAT responsive elements, operably linked to a reporter gene; and
a polynucleotide encoding a CD16A-FcεR1γ fusion which is operably linked to a promoter which causes expression of the fusion in the cell;
   e.g., wherein the fusion is capable of interacting with an antibody or antigen-binding fragment thereof that is complexed with an antigen, e.g., wherein the fusion is expressed one the host cell surface.
   (ii) exposing the host cell to a test antibody or antigen-binding fragment thereof complexed with an antigen;
   e.g., wherein the antigen is expressed on a cell surface or the antigen is isolated and immobilized on a solid substrate; and,
   (iii) determining if the reporter gene is expressed;
   e.g., wherein expression of the reporter gene is determined by detecting the activity of a polypeptide encoded by the reporter gene (e.g., luminescence, fluorescence or substrate catalysis);
   wherein the test antibody or antigen-binding fragment thereof is determined to cause antibody-dependent cell-mediated cytotoxicity (ADCC) upon binding of an antigen if reporter expression is determined, e.g., wherein reporter expression is higher than what is observed in the absence of the test antibody or antigen-binding fragment thereof or than what is observed in the presence of an antibody or antigen-binding fragment thereof (or other substance) which is known to not induce detectable or significant levels of ADCC upon antigen binding.

In an embodiment of the invention, the method further comprises the following negative-control:

(i) introducing, into an isolated host cell:
a polynucleotide comprising a promoter that comprises one or more NFAT responsive elements, operably linked to a reporter gene; and
a polynucleotide encoding a CD16A-FcεR1γ fusion which is operably linked to a promoter which causes expression of the fusion in the cell;
   e.g., wherein the fusion is capable of interacting with an antibody or antigen-binding fragment thereof that is complexed with an antigen, e.g., wherein the fusion is expressed one the host cell surface.
   (ii) not exposing the host cell to an antibody or antigen-binding fragment thereof or exposing the host cell to a negative-control antibody or antigen-binding fragment thereof complexed with an antigen or other substance which antibody or fragment is known not to induce detectable ADCC upon antigen binding; and
   (iii) determining if the reporter gene is expressed;
   wherein the test antibody or antigen-binding fragment thereof is determined to cause antibody-dependent cell-mediated cytotoxicity (ADCC) upon binding of an antigen if reporter expression in the presence of the test antibody or fragment is higher than what is observed in the absence or the antibody or antigen-binding fragment thereof or than what is observed in the presence of the negative-control antibody or antigen-binding fragment thereof (or other substance) which is known to not induce detectable or detectable levels of ADCC upon antigen binding.

In an embodiment of the invention, the method further comprises the following positive-control:

(i) introducing, into an isolated host cell:
a polynucleotide comprising a promoter that comprises one or more NEAT responsive elements, operably linked to a reporter gene; and
a polynucleotide encoding a CD16A-FcεR1γ fusion which is operably linked to a promoter which causes expression of the fusion in the cell;
   e.g., wherein the fusion is capable of interacting with an antibody or antigen-binding fragment thereof that is complexed with an antigen, e.g., wherein the fusion is expressed one the host cell surface.
   (ii) exposing the host cell to an antibody or antigen-binding fragment thereof complexed with an antigen which antibody or fragment is known to induce ADCC upon antigen binding; and,
   (iii) determining if the reporter gene is expressed;
   wherein the assay is determined to be operating if reporter gene expression is detected.

The present invention also provides a method for determining if an antibody or antigen-binding fragment thereof exhibits ADCC comprising:

1) Providing a target cell (e.g., SKOV3 cell) that expresses an antigen on the cell surface to which the antibody or fragment binds specifically; e.g., which is grown overnight at 37° C.
2) Preparing a serial dilution of antibodies to be evaluated and adding antibodies at the serial concentrations that were prepared to the target cells and, optionally, incubating, e.g., 30 min @ 37° C.;
3) Adding reporter cells e.g., including a promoter having one or more NEAT responsive elements operably linked to a beta-lactamase gene and e.g., having a CD16A^{158V}-FcεR1γ at the cell surface) to wells; and, optionally, incubating, e.g., 4 hours at 37° C.;
4) Adding CCF2-AM substrate to the cells and, optionally, incubating, e.g., 90 minutes at room temperature (e.g., about 23-25° C.); and
5) Determining if reporter gene expression has occurred by exciting the cells or a fraction thereof with light at a wavelength of about 409 nm and determining if light at a wavelength of about 460 nm to about 530 nm is emitted;
wherein the antibody or antigen-binding fragment thereof is determined to cause antibody-dependent cell-mediated cytotoxicity upon binding of an antigen if said light having a wavelength of about 460 nm to about 530 nm is detected.

The term “antigen” includes any antigen recognized by an antibody or antigen-binding fragment thereof including, for example, polypeptides such as, for example, CD20, NPC1L1, Blys, TRAIL, EGF, HER2, HERS, PCSK9, VEGF, EGFR, VEGFR, MIP3alpha, IGF1R, RANK, RANKL, or tumor necrosis factor alpha precursor, e.g., wherein the antigen is bound to a solid substrate or located on a cell surface.

The term “signal”, in relation to a reporter, refers to the indicia of expression of the reporter or the presence of the reporter's gene product (e.g., protein or mRNA) in a sample. For example, emission of light at a wavelength of about 460 nm to about 530 nm from a cell or fraction thereof comprising beta-lactamase and CCF2-AM when excited with light at a wavelength of about 409 am, or a “band” on photographic film generated during a northern blot procedure is a signal indicating the presence of a gene's RNA transcript. In an embodiment of the invention, the expression driven from a given promoter fused to a given reporter is measured by determining the signal from the reporter. In an embodiment of the invention, the signal is not cytotoxicity or is not cytokine production.

Other reporter genes may be used to indicate NFAT-dependent expression. For example, the β-galactosidase (lacZ) gene can, be operably associated with a promoter comprising one or more NFAT responsive elements. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a lea reporter gene; and methods of use thereof, e.g., as is discussed herein.

Firefly luciferase is an example of a reporter that can be operably associated with a promoter comprising one or more NFAT responsive elements. The firefly luciferase may also be altered as described in Leskinen et al. (Yeast. 20(13):1109-1113 (2003)) wherein the carboxy-terminal peroxisomal targeting signal, Ser-Lys-Leu (slk), of the firefly luciferase gene was removed. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a luciferase reporter gene; and methods of use thereof, e.g., as is discussed herein.

Other versions of luciferase that can be operably associated with a promoter comprising one or more NFAT responsive elements include the Vibrio harveyi luxA (Genbank Accession No. M10961) and Vibrio harveyi luxB (Genbank Accession No. M10961.1) genes, together, unfused or fused (i.e., luxAB or luxBA), Vibrio harveyi luxC, Vibrio harveyi luxD, Vibrio harveyi luxE, Vibrio harveyi luxCDABE, the Photorhabdus luminescens LuxCDABE operon (Genbank Accession No. M62917), Photorhabdus luminescens LuxA, Photorhabdus luminescens LuxB, Photorhabdus luminescens LuxC, Photorhabdus luminescens LuxD, Photorhabdus luminescens LuxE, optionally expressed in the presence of the Vibrio fischeri flavin oxidoreductase gene (frp; see Gupta et al., FEMS Yeast Res. 4(3):305-13 (2003)); Vibrio fischeri luxAB (see, e.g., Yang et al., FEMS Microb. Lett. 176(1): 57-65 (1999)), the Vibrio fischeri LuxCDABE operon (see e.g., Van Dyk et al., Appl. Environ. Microbiol. 60:1414-1420 (1994); Belkin et al., Appl. Environ. Microbiol. 62:2252-2256 (1996); Belkin et al., Water Res. 31:3009-3016 (1997); Genbank Accession No. AF170104); Vibrio fischeri luxA, Vibrio fischeri luxB, Vibrio fischeri luxC, Vibrio fischeri luxD or Vibrio fischeri luxE. Furthermore, the NFAT response element can be operably associated with a green fluorescent protein (GFP)-luxAB hybrid gene. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to any of such luciferase reporter genes; and methods of use thereof, e.g., as is discussed herein

The Ca²⁺ dependent photoprotein Aequorin from Aequorea victoria (Campbell, K., Chemilluminescence; Ellis Horwood: Chichester, England (1988); Ramanathan et al., Anal. Chim. Acta 369: 181-188 (1998); Witkowski et al., Anal. Chem. 66: 1837-1840 (1994); Galvan et al., Anal. Chem. 68:3545-3550 (1996)) can be operably associated with a promoter comprising one or more NFAT responsive elements. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to an aequorin reporter gene; and methods of use thereof, e.g., as is discussed herein.

The KanMX selectable marker (e.g., from Tn903) can be operably associated with a promoter comprising one or more NFAT responsive elements. Expression of the KanMX marker, in a cell, confers resistance to G418 (geniticin; Wach et al., Yeast 10:1793-1808 (1994)). Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a KanMX reporter gene; and methods of use thereof, e.g., as is discussed herein.

The pat1 (phosphinothricin N-acetyl-transferase) selectable marker can be operably associated with a promoter comprising one or more NFAT responsive elements. Expression of the pat1 marker, in a cell, confers resistance to bialaphos. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a pat1 reporter gene; and methods of use thereof, e.g., as is discussed herein.

The nat1 (nourseothricin N-acetyl-transferase) selectable marker can be operably associated with a promoter comprising one or more NFAT responsive elements. Expression of the nat1 marker, in a cell, confers resistance to nourseothricin. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a nat1 reporter gene; and methods of use thereof, e.g., as is discussed herein.

The hph (hygromycin B phosphotransferase) selectable marker can be operably associated with a promoter comprising one or more NFAT responsive elements. Expression of the nat1 marker, in a cell, confers resistance to hygromycin B. Accordingly, the present invention includes isolated host cells comprising CD16A, or a functional variant thereof, fused to FcεR1γ, or a functional variant thereof, on the surface of said cell and a polynucleotide comprising a promoter that comprises one or more NEAT responsive elements, operably linked to a hph reporter gene; and methods of use thereof, e.g., as is discussed herein.

The Sh ble selectable marker can be operably associated with a promoter comprising one or more NFAT responsive elements. Expression of the Sh ble marker, in a cell, confers resistance to Zeocin™ (Phleomycin D1; Johansson & Hahn-Hagerdal, Yeast 19: 225-231 (2002)). Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a Sh ble reporter gene; and methods of use thereof, e.g., as is discussed herein.

Other reporter genes which can be operably associated with a promoter comprising one or more NFAT responsive elements include beta-lactamase. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a beta-lactamase reporter gene; and methods of use thereof, e.g., as is discussed herein.

Furthermore, the E. coli beta-glucuronidase gene (GUS) can be operably associated with a promoter comprising one or more NFAT responsive elements. Accordingly, the present invention includes isolated host cells comprising CD16A, or a functional variant thereof, fused to FcεR1γ, or a functional variant thereof, on the surface of said cell and a polynucleotide comprising a promoter that comprises one or more NEAT responsive elements, operably linked to a GUS reporter gene; and methods of use thereof, e.g., as is discussed herein.

CAT radioassays are described, for example, by Sleigh (Anal. Biochem. 156(1):251-256 (1986)) and a non-radioactive CAT assay is described by Young et al. (Anal. Biochem. 197(2):401-407 (1991)). The chloramphenicol acetyl transferase gene can be operably associated with a promoter comprising one or more NFAT responsive elements. Accordingly, the present invention includes isolated host cells comprising CD16A, or a functional variant thereof, fused to FcεR1γ, or a functional variant thereof, on the surface of said cell and a polynucleotide comprising a promoter that comprises one or more NFAT responsive elements, operably linked to a CAT reporter gene; and methods of use thereof, e.g., as is discussed herein.

Expression of reporters, such as green fluorescent protein, luciferase or γ-galactosidase (lacZ) or any other reporter mentioned herein, can be easily determined using any of the numerous assays which are conventional and very well known in the art. For example, Billinton et al. (Biosens. Bioelectron. 13(7-8): 831-8) describe the development of a green fluorescent protein reporter in yeast. Dixon et al. (J. Steroid Biochem. Mol. Biol. 62(2-3): 165-71) describe an assay for determination of lacZ expression. Greer et al. (Luminescence 17(1): 43-74 (2002)) reviews the use of luciferase in expression assays. Leskinen at al. (Yeast 20(13): 1109-13 (2003)) describes a one-step measurement of firefly luciferase activity. Marathe et al. (Gene 154(1): 105-7 (1995)) and Gallagher et al., (“Quantiation of GUS activity by fluorometry” In: GUS Protocols: Using GUS gene as a reporter of gene expression. Academic Press, San Diego, Calif. (1992), pp. 47-59) discloses methods for assaying GUS reporter gene expression. Pignatelli et al. (Biotechnol. Appl. Biochem. 27(Pt 2): 81-88 (1998)) describe the expression and secretion of β-galactosidase. Srikantha et al., (J Bacterial 178(1): 121-9 (1996)) describes the use of Renilla reniformis luciferase. Vanoni et al. (Biochem Biophys Res Commun 164(3): 1331-8 (1989)) describes the use of E. coli β-galactosidase.

### EXAMPLES

The present invention is intended to exemplify the present invention and not to be a limitation thereof. Any method or composition disclosed below falls within the scope of the present invention.

### Example 1

### CD16 Reporter Assay (Protocol 1)—Reporter Assay Format for Use with Target Cells and Jurkat/NFat-Bla/CD16 Cells

Classical ADCC assays are set up using “Target cells” (expressing an antigen of interest), labeled with Europium-ligand or Cr⁵¹, and incubated in presence of antibody and “Effector cells” (natural killer cells or PBMCs). Upon incubation of the cells and antibody, binding of CD16A (FcγRIIIA) receptor on the effector cells to the Fc portion of the antibody molecule occurs, and a series of events ensues ending in lysis of the target cells and cellular release of the label, which is then quantitated.

Conversely, the CD16A Reporter Assay of the present invention is a surrogate assay in which the same “Target cells” can be used but these cells no longer need to be radiolabeled. The “Effector cells” in the reporter assay are Jurkat/NFAT-bla cells stably transfected with the CD16A receptor. Incubation of the Target and Effector cells with antibody allows the CD16A receptor on the Jurkat/NFAT-bla/CD16A cells to bind to the Fc portion of the antibody and activation of the NEAT sequence driving expression of β-lactamase. Cells are labeled using a fluorescent substrate for β-lactamase, CCF2-AM, which shifts from green to blue fluorescence upon signaling through CD16A, change that can be quantitated.

The ADCC Reporter Assay of the present invention is a surrogate assay in which “Effector cells” in the reporter assay are a stable line, Jurkat/NFAT-bla/CD16. Target cells express the antigen of interest. Incubation of the Target and Effector cells with antibody allows the CD16 receptor on the Jurkat/NFAT-bla/CD16 cells to bind to the Fc portion of the antibody and for activation of expression mediated by the NFAT containing promoter which drives expression of β-lactamase reporter gene. Cells are labeled using a fluorescent substrate for β-lactamase, CCF2-AM, and fluorescence is quantitated.

The general protocol steps were as follows:

1) Seeded target cells (SKOV3) in 80 μl/well, incubated overnight at 37° C.
On the following day:
2) Prepared 10× dilutions of antibodies in 96-well plate and transfer 10 μl/well to SKOV3 cells.
3) Added 10 μl of 10× concentration of Jurkat/NFAT-bla/CD16 cells/well to 96-well plates.
4) Incubated 4 hours at 37′C.
5) Added 20 μl/well of 6×CCF2-AM cell-loading substrate.
6) Incubated plates at room temperature in dark for 60-90 minutes.
7) Read fluorescence PE-Envision. (Ex=409 nm; Em=460 nm and 530 nm).
Materials Used were as Follows

### 1) Cells: Jurkat/NFAT-bla/CD16^{158V}

SKOV3 (target cells)
   2) Media: DMEM (−)phenol red
RPMI (−)phenol red
Fetal Bovine Serum (heat-inactivated)
Dialyzed Fetal Bovine Serum
Hepes Solution (1M)
L-Glutamine (200 mM)
Zeocin (100 mg/ml)
Hygromycin B (50 mg/ml)
Dulbecco's PBS w/o Ca⁺² and Mg⁺² (DPBS)
Trypsin EDTA (0.25%)

### 3) Misc: CCF2-AM Loading Kit

96-well Black Clear-bottom plates
   Media was prepared and used as follows.
   Note: phenol red-free media is used for both maintenance and use in reporter assay

A.) Media for maintenance of Jurkat/NFAT-bla/CD16 clones:
RPMI
10% heat-inactivated FBS
2 mM L-glutamine
10 mM Hepes
400 μg/ml Hygromycin B
50 μg/ml Zeocin

Cells were split 2× per week, maintaining density between 2×10⁵/ml and 1.0×10⁶/ml.

B.) Media for maintenance of SKOV3 cells:
DMEM
10% heat-inactivated FBS
2 mM L-glutamine

Cells were split 2× per week at 1:4 and 1:8 split ratios.

C.) Media for Reporter Assay:
DMEM
5% Dialyzed-FBS
   The specific steps taken in performing the assay were as follows:

### Day 1 (Day Prior to Running Assay):

1.) Trypsinized SKOV3 cells and perform cell count.

2.) Number of cells needed per plate=8 ml at 1.88×10⁵/ml

Final cell number per well=1.5×10⁴

3.) Added 80 μl/well to 96 well microtiter plate columns 1-11
96 well microtiter place column 12 was used for “No cell” and “Effector cell only” controls

4.) incubated overnight at 37° C. 15% CO₂.

### Day 2: Setting up ADCC Reporter Assay:

### A. Preparation of Antibody Dilutions:

1.) In a separate 96-well polypropylene plate, prepared 10× concentrations of the antibodies to be tested as outlined below. Each dilution series was run in three-fold titrations and run in triplicate on the test plates.
Determined the number of antibodies and plates to be run in assay.

Plate Layout

| | 1 | 2 | 3 | 4 | 5 | 6 | 7 | 8 | 9 | 10 | 11 | 12 |
|---|---|---|---|---|---|---|---|---|---|---|---|---|
| trastuzumab | 10 | 3.333333 | 1.111111 | 0.37037 | 0.123457 | 0.041152 | 0.013717 | 0.004572 | 0.001524 | 0.000508 | No Ab | Jurkat only |
| trastuzumab | 10 | 3.333333 | 1.111111 | 0.37037 | 0.123457 | 0.041152 | 0.013717 | 0.004572 | 0.001524 | 0.000508 | No Ab | Jurkat only |
| trastuzumab | 10 | 3.333333 | 1.111111 | 0.37037 | 0.123457 | 0.041152 | 0.013717 | 0.004572 | 0.001524 | 0.000508 | No Ab | Jurkat only |
| 5X mutant | 10 | 3.333333 | 1.111111 | 0.37037 | 0.123457 | 0.041152 | 0.013717 | 0.004572 | 0.001524 | 0.000508 | No Ab | Jurkat only |
| 5X mutant | 10 | 3.333333 | 1.111111 | 0.37037 | 0.123457 | 0.041152 | 0.013717 | 0.004572 | 0.001524 | 0.000508 | No Ab | No Cells |
| 5X mutant | 10 | 3.333333 | 1.111111 | 0.37037 | 0.123457 | 0.041152 | 0.013717 | 0.004572 | 0.001524 | 0.000508 | No Ab | No Cells |
| N297A | 10 | 3.333333 | 1.111111 | 0.37037 | 0.123457 | 0.041152 | 0.013717 | 0.004572 | 0.001524 | 0.000508 | No Ab | No Cells |
| N297A | 10 | 3.333333 | 1.111111 | 0.37037 | 6.123457 | 0.041152 | 0.013717 | 0.004572 | 0.001524 | 0.000508 | No Ab | No Cells |

2.) For dilutions, needed 10 μl of each 10× dilution of antibody.
For example above, added 50 μl of DMEM/5% Dialyzed FBS to wells in columns 2 through 10.
3.) Made up 250 μl of 10× (100 μg/ml) concentration of antibody as follows:
Herceptin=21 mg/ml; prepare 250 μl @100 μg/ml=1.2 μl in 249 μl media
5× herceptin mutant=1.13 mg/ml; prepare 250 μl @100 μg/ml=22 μl in 228 μl media
N297A herceptin mutant=1.31 mg/ml; prepare 250 μl @100 μg/ml=19 μl in 231 μl media

The 5× mutant of herceptin exhibits ADCC at about 5 times the level observed with herceptin. The N297A mutant exhibits a lower level of ADCC than herceptin.
4.) Added 75 μl of the diluted antibodies to wells in Column 1, as designated in plate layout. Prepared 3-fold titrations across plate by diluting 25 μl from Column 1 into 50 μl Column 2 and continued across plate to Column 10.
5.) Removed SKOV3 plates (Day 1) from incubator.
6.) Transferred 10 μl of the antibody dilutions to designated wells.
7.) Added 10 μl of media to Column 11 of each plate for “No Antibody Controls”.
8.) Added 90 μl of media to Column 12, rows A-D, for “Effector cell controls”.
9.) Added 100 μl of media to Column 12, rows E-H, for “Background Controls”.
10.) Transferred plates to incubator for 15 minutes while preparing the Jurkat/NFAT-bla/CD16 cells for assay.

### B. Preparation of Jurkat/NFAT-Bla/CD16 Cells for Addition to Plates:

1.) Cell count: (Needed 1.5×10⁷ cells per plate)
2.) Spun down cells at 1200 rpm for 5 minutes. Gently aspirated media off of the pellet.
3.) Resuspended cell pellet to 1×10⁷ cells/ml in DMEM/5% dialyzed FBS=1.5 ml.
4.) Removed SKOV3/Ab plates from incubator.
5.) Added 10 μl cells/well (1×10⁵ cells) to all rows of Columns 1-11 and row A-D of Column 12.
6.) Gently tapped plates to evenly distribute the cells in the wells.
6.) Transferred plates back to incubator and incubated for a total of 3.5-4 hours.

### C. CCF2-AM Substrate Preparation and Addition

1.) Removed assay plates from incubator and brought to room temperature.
2.) Prepared CCF2-AM substrate as follows: (need 2 ml of 6×CCF2-AM per plate)
   Added 120 μl of Solution B (from labeling kit) to 15 ml conical tube
   Added 24 μl of thawed CCF4-AM (Solution A) substrate to Solution B.
   Added 1856 μl of Solution C to the combined solutions and vortexed vigorously.
3.) Added 20 μl of 6×CCF2-AM substrate solution to each well.
4.) Covered plates with foil and incubate at room temperature, in dark, for 60-90 minutes.
5.) Conversion of Blue and Green fluorescence was monitored using an epifluorescence microscope fitted with B-lac filter set.
   Excitation filter: HQ405/20X (405+/−10)
   Dichroic mirror: 425 DCXR
   Emission filter: HQ435LP

### D. Fluorescence Detection:

1.) Plates were read (bottom-read) on PE Envision instrument fitted with the following filters:
   Excitation filter: HQ405/20X (405+/−10 nm)
   Emission filter: HQ460/40m (460+/−20 nm) (Blue)
   Emission filter: HQ530/30m (530+/−15 nm) (Green)

### Data Calculations:

Averaged the blank wells (12E-12H) for both Blue and Green channel reads.
1.) Calculated Blue (“Background-subtracted”)=Average of Blank Blue values Experimental Blue values.
2.) Calculated Green (“Background-subtracted”)=Average of Green Blank values Experimental Green values.
3.) Calculated Blue/Green Ratio=Divide Blue (background-subtracted) by Green (background-subtracted).
4.) Averaged the Blue/Green Ratio for the wells containing “No-Ab Control”.
5.) Calculated the Response Ratio: divide Blue/Green Ratio by the average of “No-Ab Control” wells.
6.) Plotted the values and determined EC₅₀ values using a four-parameter fit (graphing software, e.g., Prism).
   *The “No Antibody Control” represented wells containing target and effector cells, but no antibodies. The “Background control” represented the wells with Media only (no cells).

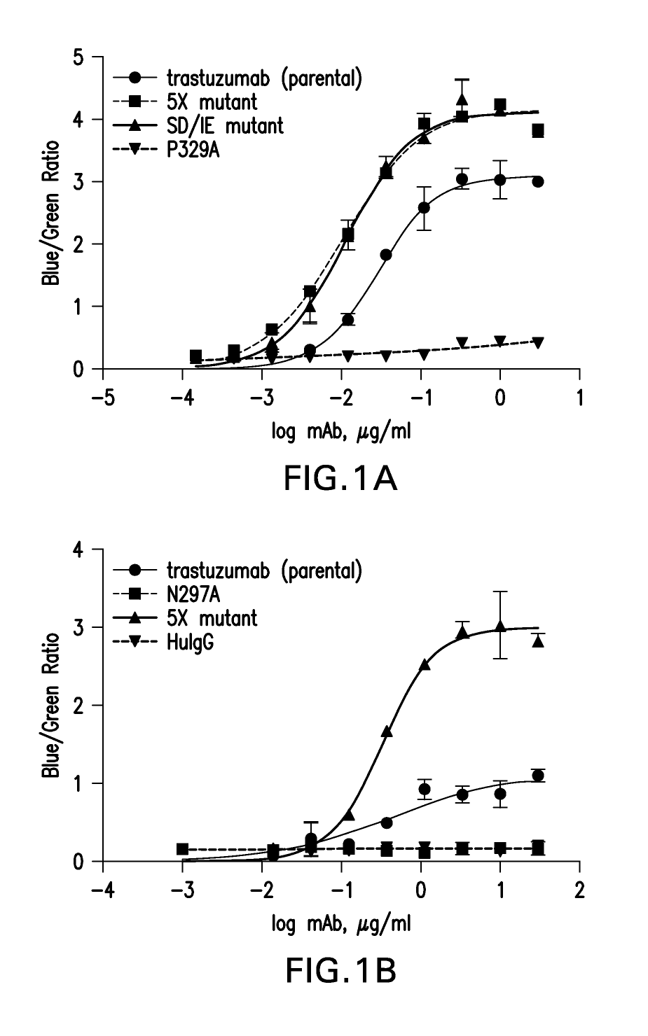

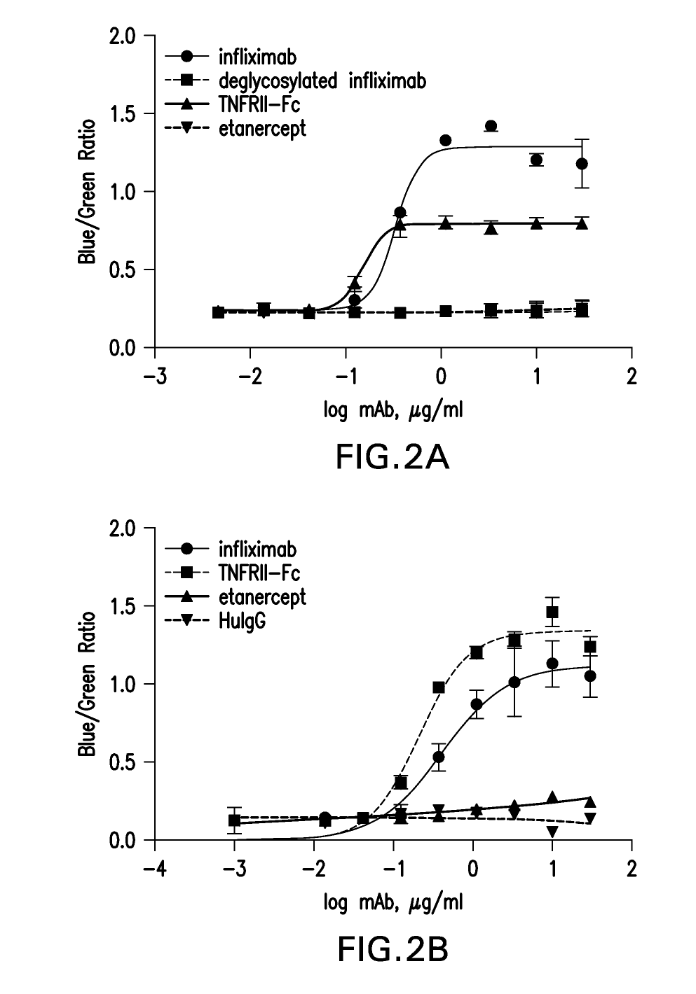

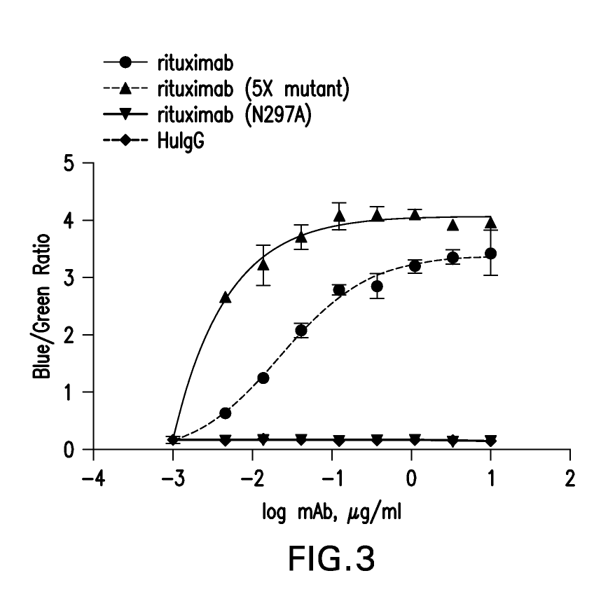

The ability of the CD16 Reporter cell-based assay to serve as a surrogate assay for “Classical” ADCC assays was determined by testing the reactivity of different antibodies with their respective target-expressing cells and the Jurkat/NFAT-bla/CD16A reporter cells. In FIG. 1A, the assay was set up to compare the reactivity of trastuzumab (wild-type antibody) and different variants in the CD16 Reporter cell-based assay, using target cells expressing Her2 (SKOV3) and the Jurkat/NFAT-bla/CD16A reporter cells. The results of this assay indicated that the parental trastuzumab showed lower signaling of the NFAT-bla expression than both the 5× mutant and the SD/IE mutant did. In addition, the P329A antibody showed very little, if any, signaling in the assay. These results are consistent with published reports for “classical” ADCC assays with these antibodies. In FIG. 2A, the reactivity of the anti-TNFα antagonists, infliximab, etanercept and TNFRII-Fc fusion protein were tested in the reporter assay, using HEK293Flpln cells expressing membrane-bound TNFα and the Jurkat/NFAT-bla/CD16A reporter cells. Results of this assay showed that infliximab showed greater reactivity in the assay than the TNFRII-Fc fusion protein. Both the commercial etanercept and the deglycosylated infiximab both showed no reactivity in the assay. In FIG. 3, the CD16 Reporter cell-based assay was used to compare the reactivity of different anti-CD20 antibodies, using Raji cells as the target-expressing cell line. In this assay, the panel of antibodies (wild-type and variants) is similar to that used in FIG. 1A for Her2 cells. The results of the assay run in FIG. 3 indicated that the wild-type rituxumab showed less reactivity than that seen with the 5× mutant rituxumab. Both the N297A mutant, which abrogates FcγR binding, and HuIgG, showed no reactivity in the assay.

### Example 2

### CD16A Reporter Assay (Protocol 2)-Reporter Assay Format for Use with Purified Target Protein (Instead of Target Cells)

This protocol, at difference of the Protocol version 1, describes an assay using, as a target (an antigen of interest), an antibody-bound recombinant protein (i.e., receptor) instead of target cells expressing that specific receptor on the cell membrane. This method would be advantageous in cases where cells expressing a specific target receptor are not available.

The general protocol steps were as follows:

### Day 1

1. Coated 96-well plates with target protein and incubated plate(s) overnight at 4° C.

### Day 2:

2. Washed plates 3× with DPBS.
3. Blocked plates for 60-90 minutes with 3% BSA/DPBS, at room temperature
4. Washed plates 3× with DPBS.
5. While plates were blocking, prepared 1× dilutions of antibodies/controls in a 96-well plate and then transferred 100 μl of each dilution to respective wells in the plates from step 4 (above).
6. Incubated plates at room temperature for 1 hour.
7. Washed plates 3× with DPBS.
8. Added 100 μl of Jurkat/NFAT-bla/CD16A cells (to plates from step 7).
9. Incubated 3.5 to 4 hours at 37° C.
10. Added 20 μl/well of 6×CCF2-AM cell-loading substrate.
11. Incubated plates at RT in dark for 60-90 minutes.
12. Read fluorescence PE-Envision. (Ex=409 nm; Em=460 nm and 530 nm)
   Materials Used were as Follows

| | | |
|---|---|---|
| 1. Cells: | Jurkat/NFAT-bla/CD16A^{158V} | Clone 1F12 (passage #) |
| 2. Media: | RPMI (−)phenol red | Invitrogen Cat#11835 |
| | Fetal Bovine Serum | Sigma Cat#F4135 |
| | (heat-inactivated) | |
| | Dialyzed Fetal Bovine Serum | Invitrogen Cat#26400 |
| | Hepes Solution (1M) | Invitrogen Cat#15630 |
| | L-Glutamine (200 mM) | Mediatech Cat#MT 25005CI |
| | Zeocin (100 mg/ml) | Clontech Cat#R250-01 |
| | Hygromycin B (50 mg/ml) | Mediatech Cat#MT 30240CR |
| | Dulbecco's PBS w/o Ca⁺² | Mediatech Cat#MT21031CV |
| | and Mg⁺² (DPBS) | |
| | Bovine Serum Albumin | Santa Cruz Cat#sc-2323 |
| 3. Misc: | CCF2-AM Loading Kit | Invitrogen Cat#K1032 |
| | 96-well Black Clear- | Costar Cat#3603 |
| | bottom plates | |
| | 96-well polypropylene plates | Costar Cat#3790 |
| | Her2 ECD protein | 0.4 mg/ml |
| | (purified in-house) | |

### Media was Prepared and Uses as Follows.

Note: phenol red-free media is used for both cell maintenance and use in reporter assay

A. Media for maintenance of Jurkat/NFAT-bla/CD16A clones:
RPMI
10% heat-inactivated FBS
2 mM L-glutamine
10 mM Hepes
400 μg/ml Hygromycin B
50 μg/ml Zeocin

Cells were split 2× per week, maintaining density between 2×10⁵/ml to 1×10⁶/ml.

B. Media for Reporter Assay:
RPMI
5% Dialyzed FBS

C. 3% BSA in DPBS (Filter-Sterilized and Store at 4° C.)

The Specific Protocol Used was as Follows:

### Day 1 (Day Prior to Running Assay):

1.) Made up dilution of Her2 ECD in DPBS.
2.) Her2 ECD=0.4 mg/ml; make up 10 ml @1 μg/ml=25 μl/10 ml DPBS
3.) Added 100 μl/well to Costar 3603 plate as outlined below:
   a. Column 12=no protein (blank wells)
   b. Columns 1-11=100 μi/well of diluted Her2 ECD
4.) Covered plate with plate sealer.
5.) Incubated plates overnight at 4° C.

### Day 2: Setting Up Reporter Assay:

1.) Removed proteins from plate by dumping the liquid in biohazard waste container.
2.) Washed plates 3× with DPBS, dumping buffer in biohazard waste box with paper towels. Gently rapped plate on paper towels at end of final wash to remove residual buffer.
3.) Blocked non-specific reactivity by adding 250 μl/well of 3% BSA/DPBS. Incubated plate for 1.5-2 hours at room temperature.
4.) Prepared antibody dilutions-
   Approximately 30 minutes prior to the end of the blocking period, prepared 1× concentrations of the antibodies to be tested, as outlined in the table below, in a separate 96-well polypropylene plate (item 5). Each dilution series was run in three-fold titrations and run as outlined below. Determined number of antibodies and plates to be run in the assay.

| | trastuzumab | trastuzumab | trastuzumab | GFI5.0 | GFI5.0 | N297A | N297A | 5X mutant | 5X mutant | HuIgG | | |
|---|---|---|---|---|---|---|---|---|---|---|---|---|
| | 1 | 2 | 3 | 4 | 5 | 6 | 7 | 8 | 9 | 10 | 11 | 12 |
| A | 30 μg/ | 30 μg/ | 30 μg/ | 30 μg/ | 30 μg/ | 30 μg/ | 30 μg/ | 30 μg/ | 30 μg/ | 30 μg/ | No Ab | Media only |
| | ml | ml | ml | ml | ml | ml | ml | ml | ml | ml | | |
| B | 10.000 | 10.000 | 10.000 | 10.000 | 10.000 | 10.000 | 10.000 | 10.000 | 10.000 | 10.000 | No Ab | Media only |
| C | 3.333 | 3.333 | 3.333 | 3.333 | 3.333 | 3.333 | 3.333 | 3.333 | 3.333 | 3.333 | No Ab | Media only |
| D | 1.111 | 1.111 | 1.111 | 1.111 | 1.111 | 1.111 | 1.111 | 1.111 | 1.111 | 1.111 | No Ab | Media only |
| E | 0.370 | 0.370 | 0.370 | 0.370 | 0.370 | 0.370 | 0.370 | 0.370 | 0.370 | 0.370 | No Ab | Media only |
| F | 0.123 | 0.123 | 0.123 | 0.123 | 0.123 | 0.123 | 0.123 | 0.123 | 0.123 | 0.123 | No Ab | Media only |
| G | 0.041 | 0.041 | 0.041 | 0.041 | 0.041 | 0.041 | 0.041 | 0.041 | 0.041 | 0.041 | No Ab | Media only |
| H | 0.014 | 0.014 | 0.014 | 0.014 | 0.014 | 0.014 | 0.014 | 0.014 | 0.014 | 0.014 | No Ab | Media only |

For dilutions, will need 120 μl of each 1× dilution of antibody/well so that there is enough volume to transfer 100 μl of each dilution to the Her2 ECD-coated assay plate

Thus,
a. Added 120 μl of RPMI/5% dialyzed-FBS to wells in the corresponding rows B through H of the 96-well plate.
b. Made up outlined volume of 30 μg/ml concentration of antibody as follows:

Herceptin=21 mg/ml
Prepared 1:10 dilution in RPMI/5% dialyzed-FBS=2.1 mg/ml
From 2.1 mg/ml; prepared 1000 0 @30 μg/ml=14 d in 986 μl media 5× mutant=1.13 mg/ml; prepared 400 0 @30 μg/ml=11 μl in 389 μl media N297A mutant=1.31 mg/ml; prepared 400 μl @30 μg/ml=9 μl in 391 μl media GFI5.0 mutant=7.9 mg/ml; prepared 1000 0 @30 μg/ml=4 μl in 996 μl media HuIgG=11 mg/ml; prepared 1000 μl @30 μg/ml=3 μl in 997 μl media
c. Added 200 μl of the diluted antibodies to wells in Row A, as designated in plate layout. Prepared 3-fold titrations down plate by diluting 60 μl from Row A into 120 μl in Row B and continued down plate to Row H.
d. For controls in Columns 11 and 12, used 120 μl RPMI/5% dialyzed-FBS/well
5.) At end of blocking incubation in step 3, dumped out BSA block.
   Washed plates 3× with DPBS, as outlined in step 2 above.
6.) Transferred 100 μl of diluted antibodies and control media to wells in plate from step 5, as outlined in above plate grid.
7.) Incubated plates at room temperature for 1 hour.
8.) Preparation of Jurkat/NFAT-bla/CD16A cells
   a. Cell count: 1.5×10⁷ cells per plate
   b. Determined number of plates to be run and multiplied by 1.5×0⁷ cells/plate.
      For example: if 5 plates are needed,
      Total number of reporter cells needed=1.5×10⁷ cells/plate×5=7.5×10⁷ cells
   c. Spun down cells at 1200 rpm for 5 minutes. Gently aspirated media off of pellet.
   d. Resuspended cell pellet (i.e., 7.5×10⁷ cells) to 1×10⁶ cells/ml in 75 ml
      RPMI/5% dialyzed FBS (i.e., Volume of media=# total cells/final cell concentration).
      For example: Volume of media=7.5×10⁷ cells/1×10⁶ cells/ml=75 ml
9.) At the end of the antibody incubation period in step 7, removed antibodies from the wells.
10.) Washed plates 3× with DPBS as previously outlined in step 2.
11.) Added 100 μl of Jurkat/NFAT-bla/CD16A cells/well to all rows of Columns 1-11.
12.) Transferred plates to 37° C., 5% CO₂ incubator and incubated for a total of 3.5-4 hours
13.) Prepared and added CCF2-AM Substrate
   a. Removed assay plates from incubator and brought to room temperature.
   b. Prepared CCF2-AM substrate as follows: (need 2 ml of 6×CCF2-AM per plate)
      Added 120 μl of Solution B (from labeling kit) to 15 ml conical tube.
      Added 24 μl of thawed CCF2-AM (Solution A) substrate to Solution B.
      Added 1856 μl of Solution C to the combined solutions and vortexed vigorously.
   c. Added 20 μl of 6×CCF2-AM substrate solution to each well.
14.) Covered plate(s) with foil and incubated at room temperature, in dark, for 60-90 minutes.
15.) Fluorescence Detection: Conversion of Blue and Green fluorescence was monitored using epifluorescence microscope fitted with β-lac filter set.
   Excitation filter: HQ405/20X (40541-10)
   Dichroic mirror: 425 DCXR
   Emission filter: HQ435LP

Plates were read (bottom-read) on PE Envision instrument fitted with the following filters:
Excitation filter: HQ405/20X (405+/−10 nm)
Emission filter: HQ460/40m (460+/−20 nm) (Blue)
Emission filter: HQ530/30m (530+/−15 nm) (Green)

### Data Calculations:

1. Averaged the blank wells (12E-12H) for both Blue and Green channel reads.
2. Calculated Blue (“Background-subtracted”)=Average of Blank Blue values Experimental Blue values.
3. Calculated Green (“Background-subtracted”)=Average of Green Blank values Experimental Green values.
4. Calculated Blue/Green Ratio=Divide Blue (background-subtracted) by Green (background-subtracted).
5. Averaged the Blue/Green Ratio for the wells containing “No-Ab Control”.
6. Calculated the Response Ratio: divide Blue/Green Ratio by the average of “No-Ab Control” wells.
7. Plotted the values and determine EC₅₀ values using a four-parameter fit (graphing software, e.g., Prism).

The “No Antibody Control” (No-Ab Control) represented wells containing target protein (Her2 ECD) and Jurkat/NFAT-bla/CD16A cells, but not antibodies. The “Background control” represented the wells with Media only (no cells and no Her2 ECD).

The ability of the CD16A Reporter Assay to be used with purified target protein in place of target-expressing cell lines was tested in assays for Her2 and TNFalpha antibodies to assess the usefulness of the assay. In FIG. 1B, the assay was set up to compare the reactivity of trastuzumab (wild-type antibody) and different variants in the CD16 Reporter cell-based assay, using purified Her2 ECD-coated plates in place of the target-expressing cell line, SKOV3. The results of this assay are similar to that seen in FIG. 1A, with the SKOV3 cells. The results indicated that WT trastuzumab had moderate reactivity in the assay while the 5× mutant had much greater reactivity. Both the non-binding mutant, N297A and the control antibody, HuIgG, showed no reactivity. In FIG. 2B, the assay was set up to test anti-TNFalpha antagonists, infliximab, etanercept and TNFRII-Fc, using purified human TNFα in place of the TNFα-expressing Jurkat cell line used in FIG. 2A. The results of the assay in FIG. 2A are slightly different than that seen with the assay run with the target-expressing cells. In the assay configuration using purified TNFα, the TNFRII-Fc fusion protein has greater reactivity than does infliximab, whereas the opposite was true for the assay run with the Jurkat/TNFα cell line. Etanercept showed slight reactivity in this assay format, as opposed to being non-reactive in the assay run with target cells.

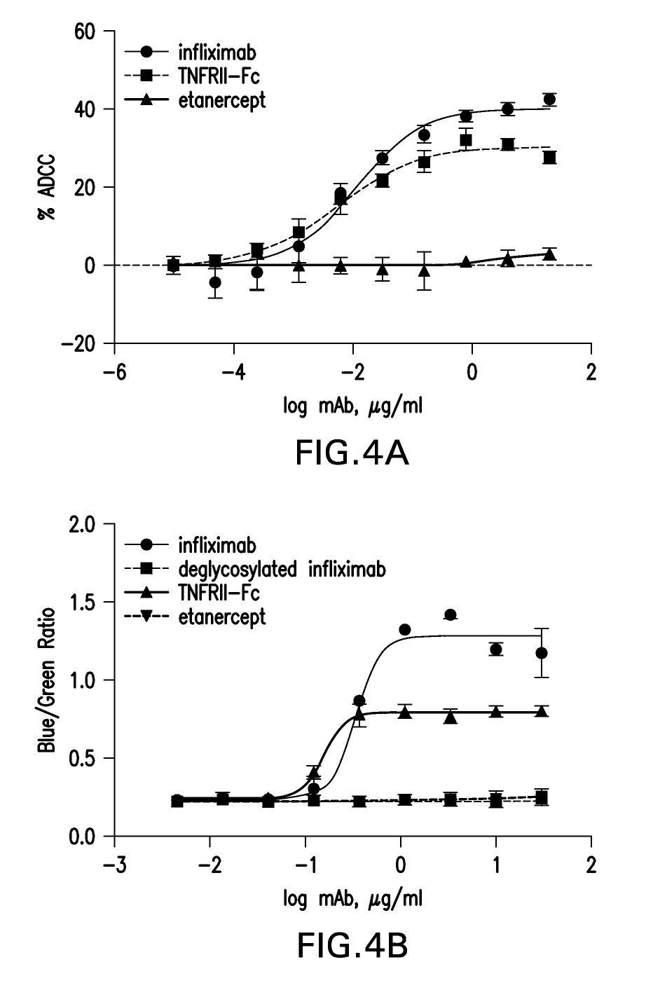

In FIG. 4A, the activity of the anti-TNFα antagonists, infliximab, etanercept and TNFRII-Fc was assessed in a classical ADCC assay, using Jurkat cells expressing a membrane-bound mutant of TNFalpha (delta1-12) as the target cells and primary NK (natural killer) cells, expressing FcγRIIIA, as the effector cells.

The ADCC activity of the antibodies, seen in FIG. 4A, was compared to the activity of the same anti-TNRc antagonists in a CD16A Reporter assay in FIG. 46. In the reporter assay, the target cells used were HEK 293Flpin cells, expressing a membrane-bound mutant of TNFalpha (delta1-12) and the effector cells are Jurkat/NFAT-bla/CD16A. The ability of the antibodies to elicit signaling in the CD16A reporter assay was similar to the increases in cellular cytotoxicity that these antibodies elicit in the classical ADCC assay. In both assays, infliximab had the highest activity, followed by lower activity with TNFRII-Fc and etanercept was inactive.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. An isolated host cell comprising CD16A, or a functional variant thereof, fused to FcεR1γ, or a functional variant thereof, on the cell surface and a polynucleotide comprising a promoter that comprises one or more NFAT responsive elements, operably linked to a reporter gene.

2. The isolated host cell of claim 1 wherein said CD16A fused to FcεR1γ is bound to an antibody or antigen-binding fragment thereof that is complexed with an antigen.

3. The host cell of claim 1 wherein the antigen is CD20, NPC1L1, Blys, TRAIL, EGF, HER2, HERS, PCSK9, VEGF, EGFR, VEGFR, MIP3alpha, IGE1R, RANK, RANKL, or tumor necrosis factor alpha precursor.

4. The host cell of claim 1 wherein the fusion is CD16A^{158V}-FcεR1γ.

5. The host cell of claim 1 which is a T-lymphocyte, an immortalized T lymphocyte, a Jurkat cell, a Raji cell or a Wil-2 B-cell.

6. A method for making the host cell of claim 1; comprising introducing a polynucleotide encoding the fusion and the polynucleotide comprising the promoter operably linked to the reporter gene into an isolated host cell and culturing the host cell under conditions wherein the fusion is expressed and located on the cell surface.

7. A method for evaluating the potential, for antibody-dependent cellular cytotoxicity, of an antibody or antigen-binding fragment thereof when complexed with an antigen comprising contacting a cell line expressing CD16A fused to FcεR1γ with a complex between an antibody or antigen-binding fragment thereof and an antigen that is on cell surface or that is immobilized to a solid substrate; and measuring CD16A-mediated expression of a reporter gene operably linked to a promoter comprising one or more NFAT responsive elements in said cell; wherein the antibody or fragment is determined to exhibit said cytotoxicity if said expression is observed.

8. The method of claim 7 comprising
(1) introducing, into an isolated host cell:
(i) a polynucleotide comprising a promoter that comprises one or more NEAT responsive elements, operably linked to a reporter gene; and
(ii) a polynucleotide encoding a CD16A-FcεR1γ fusion protein which is operably linked to a promoter;
wherein the fusion, when on the host cell surface, is capable of interacting with an antibody or antigen-binding fragment thereof;
(2) exposing the host cell to an antibody or antigen-binding fragment thereof complexed with an antigen on a cell surface or immobilized to a solid substrate; and
(3) determining if expression of the reporter gene is activated;
wherein the antibody or antigen-binding fragment thereof is determined to cause said cytotoxicity if said expression is observed.

9. The method of claim 8 wherein the antibody or fragment is complexed with an antigen that is immobilized on a solid substrate.

10. The method of claim 8 wherein the antigen is immobilized on a plastic support.

11. The method of claim 8 wherein the antibody or fragment is complexed with an antigen that is located on a cell surface.

12. The method of claim 8 wherein the antigen is CD20, NPC1L1 Blys, TRAIL, EGF, HER2, HERS, PCSK9, VEGF, EGFR, VEGFR, MIP3alpha, IGF1R, RANK, RANKL, or tumor necrosis factor alpha precursor.

13. The method of claim 8 wherein the reporter gene is a beta-lactamase gene.

14. The method of claim 13 wherein beta-lactamase reporter gene activation is detected by adding CCF2-AM substrate to said isolated host cell comprising the reporter gene and determining whether said host cell fluoresces light having a wavelength in the range of about 460 nm to about 530 nm when excited with light of a wavelength of about 409 nm; wherein the antibody or fragment is determined to cause said cytotoxicity if said fluoroescence is detected.

15. The method of claim 8 wherein the host cell is a T-lymphocyte, an immortalized T lymphocyte, a Jurkat cell or a Wil-2 B-cell.

16. The method of claim 8 wherein the fusion is CD16A^{158V}-FcεR1γ.


SEQUENCE LISTING (5 sequences)

SEQ ID NO: 1 - DNA, 31 bp, Homo sapiens
ggaggaaaaactgtttcatacagaaaggcgt

SEQ ID NO: 2 - protein, 208 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGEF

SEQ ID NO: 3 - protein, 66 aa, Homo sapiens
PQLCYILDAILFLYGIVLTLLYCRLKVIQVRKAAITSYEKSDGVYTGLSTRNQETYETLKHEKPPQ

SEQ ID NO: 4 - protein, 274 aa, Homo sapiens [sequence omitted]

SEQ ID NO: 5 - DNA, 996 bp, Homo sapiens
agctctctggctaactagagaacccactgcttactggcttatcgaaattaatacgactcactatagggagacccaagctggctagcgtttaaacttaagcttggtaccgagctcggatccctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctggggaattccctcagctctgctatatcctggatgccatcctgtttctgtatggaattgtcctcaccctcctctactgtcgactgaaggtaatccaagtgcgaaaggcagctataaccagctatgagaaatcagatggtgtttacacgggcctgagcaccaggaaccaggagacttacgagactctgaagcatgagaaaccaccacagtaggcggccgctcgagtctaga